(19) United States
(12) Patent Application Publication
(10) Pub. No.: US 2024/0221163 A1
(43) Pub. Date: Jul. 4, 2024

(54) METHOD AND APPARATUS FOR MEASURING DEVIATION ANGLE OF GAZE POSITION BASED ON THREE-DIMENSIONAL RECONSTRUCTION

(71) Applicants: Zhongshan Ophthalmic Center, Sun Yat-Sen University, Guangzhou (CN); Tsinghua University, Beijing (CN)

(72) Inventors: Ruixin Wang, Guangzhou (CN); Haotian Lin, Guangzhou (CN); Feng Xu, Beijing (CN); Xinping Yu, Guangzhou (CN); Junfeng Lyu, Beijing (CN)

(21) Appl. No.: 18/221,897

(22) Filed: Jul. 14, 2023

(30) Foreign Application Priority Data
Dec. 28, 2022 (CN) 202211693689.1

(51) Int. Cl. G06T 7/00; A61B 3/08; G06T 3/60; G06T 7/73

(52) U.S. Cl. CPC G06T 7/0016; A61B 3/085; G06T 3/60; G06T 7/74; G06T 7/75; G06T 2200/04; G06T 2207/20081; G06T 2207/20084; G06T 2207/30041; G06T 2207/30201

(57) ABSTRACT

Disclosed is a method and apparatus for measuring a deviation angle of a gaze position based on 3D reconstruction. A face image sequence of a testee is acquired. Covering conditions of the face image sequence are obtained. Key frame images are determined and input into a second neural network to obtain feature point heat maps that are converted into facial feature point coordinates. An objective function is constructed. A head pose corresponding to the key frame images is obtained. An eyeball position is initialized. An eyeball rotation angle of a reference gaze position is set as a preset angle. 3D coordinates of an eyeball in a reference gaze position image in a head coordinate system is solved. 3D coordinates of an eyeball in a to-be-measured image in the head coordinate system are fixed. An eyeball rotation angle is solved. A deviation angle of a gaze position is obtained.

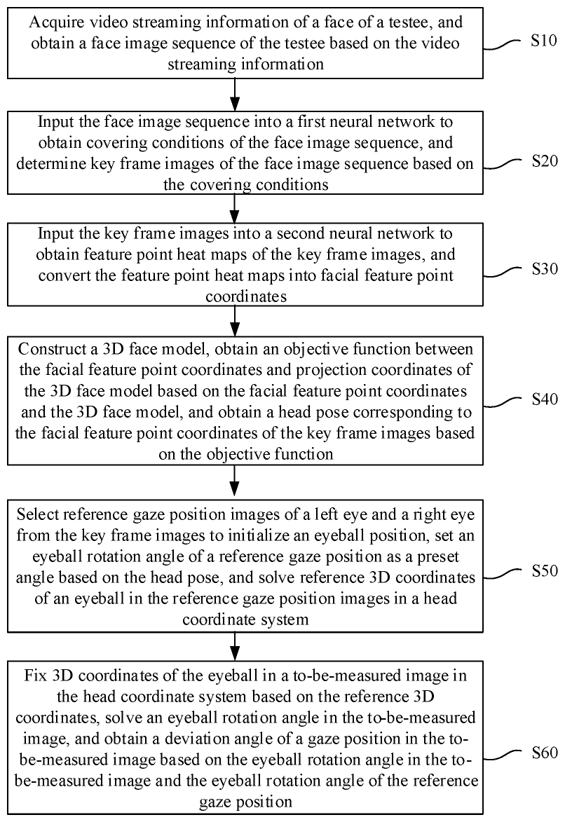

### CROSS REFERENCE TO RELATED APPLICATIONS

The present application claims the benefit of Chinese Patent Application No. 202211693689.1 filed on Dec. 28, 2022, the contents of which are incorporated herein by reference in their entirety.

### TECHNICAL FIELD

The present application relates to the technical field of gaze position measurement, and in particular, to a method and apparatus for measuring a deviation angle of a gaze position based on three-dimensional (3D) reconstruction.

### BACKGROUND

Squint, such as horizontal esotropia or vertical squint, refers to intermittent or persistent inability of both eyes of a patient to simultaneously gaze at a target. A line of sight of a squint eye cannot point to a same object seen by the other eye. Squint is a common ophthalmic problem, affecting approximately 3% to 8% of the population. A probability of squint in children is approximately 3%. Squint can be cured through early identification and intervention. However, delayed diagnosis and treatment may cause difficulties in binocular depth perception and serious psychological development disorders to children. Squint also brings many problems to adults, such as affecting reading ability, increasing a falling risk, and bringing social anxiety. This seriously affects physical and mental health and greatly reduces quality of life. Therefore, squint needs to be measured and determined in time. Measurement of squint is mainly to measure a deviation angle of a gaze position, which is of great help to subsequent squint surgery design, disease type diagnosis, disease condition estimation, and the like. At present, widely used methods for measuring a deviation angle of a gaze position mainly include a triangular prism plus cover detection method, corneal reflection method, visual field arc inspection method, and the like. However, the foregoing measurement methods depend on cooperation of patients to varying degrees, and are affected by subjective factors of examiners. There are large deviations and different examiners. Obtained inspection results are inconsistent, and objective consistency is lacking.

### SUMMARY

Embodiments of the present application provide a method and apparatus for measuring a deviation angle of a gaze position based on 3D reconstruction to improve convenience and accuracy of measuring a deviation angle of a gaze position.

According to a first aspect, the present application provides a method for measuring a deviation angle of a gaze position based on 3D reconstruction.

The present application is implemented by the following technical solution:

The method for measuring a deviation angle of a gaze position based on 3D reconstruction includes: acquiring video streaming information of a face of a testee, and obtaining a face image sequence of the testee based on the video streaming information;
inputting the face image sequence into a first neural network to obtain covering conditions of the face image sequence, and determining key frame images of the face image sequence based on the covering conditions;
inputting the key frame images into a second neural network to obtain feature point heat maps of the key frame images, and converting the feature point heat maps into facial feature point coordinates;
constructing a 3D face model, obtaining an objective function between the facial feature point coordinates and projection coordinates of the 3D face model based on the facial feature point coordinates and the 3D face model, and obtaining a head pose corresponding to the facial feature point coordinates of the key frame images based on the objective function;
selecting a reference gaze position image of a left eye and a reference gaze position image of a right eye from the key frame images to initialize an eyeball position, setting an eyeball rotation angle of a reference gaze position as a preset angle based on the head pose, and solving reference 3D coordinates of eyeballs in the reference gaze position images in a head coordinate system; and
fixing 3D coordinates of the eyeball in a to-be-measured image in the head coordinate system based on the reference 3D coordinates and solving an eyeball rotation angle in the to-be-measured image; and obtaining a deviation angle of a gaze position in the to-be-measured image based on the eyeball rotation angle in the to-be-measured image and the eyeball rotation angle of the reference gaze position.

In a preferred example of the present application, it may be further set that before the inputting the face image sequence into a first neural network to obtain covering conditions of the face image sequence, the method further includes:
training the first neural network, which specifically includes: constructing an initial first neural network, acquiring a face image sequence, and labeling real covering conditions of the face image sequence; and sequentially inputting each image in the face image sequence as a training image into the initial first neural network to obtain a predicted covering condition of the training image, constructing a cross-entropy loss function based on the predicted covering condition and a corresponding real covering condition, and performing supervised training on the initial first neural network based on the cross-entropy loss function to obtain a trained neural network as the first neural network.

In a preferred example of the present application, it may be further set that before the inputting the key frame images into a second neural network to obtain feature point heat maps of the key frame images, the method further includes:
training the second neural network, which specifically includes: constructing an initial second neural network, obtaining a face image from a public dataset, labeling real feature point coordinates on the face image, and converting the real feature point coordinates into a real feature point heat map; and inputting the face image into the initial second neural network to obtain a predicted feature point heat map of the face image, constructing an L1 loss function based on the real feature point heat map and the predicted feature point heat map, and performing supervised training on the initial second neural network based on the L1 loss function until the L1 loss function converges to obtain a trained initial second neural network as the second neural network.

In a preferred example of the present application, it may be further set that the fixing 3D coordinates of the eyeball in a to-be-measured image in the head coordinate system based on the reference 3D coordinates and solving an eyeball rotation angle in the to-be-measured image includes: fixing the 3D coordinates of the eyeball in the to-be-measured image in the head coordinate system, constructing an objective function between an iris feature point and an iris model projection point, and solving the eyeball rotation angle in the to-be-measured image through iterative optimization.

In a preferred example of the present application, it may be further set that the eyeball rotation angle of the reference gaze position is set as the preset angle, and the preset angle is set to 0 degrees.

In a preferred example of the present application, it may be further set that the acquiring video streaming information of a face of a testee includes:
disposing a first visual target at a first preset distance and a second visual target at a second preset distance in front of the testee, and acquiring, through a camera, video streaming information of the testee gazing at the first visual target and the second visual target when the left eye is covered, the right eye is covered, both the left eye and the right eye are covered, and neither the left eye nor the right eye is covered.

In a preferred example of the present application, it may be further set that the covering condition of the face image sequence is one of the following conditions: the left eye is covered, the right eye is covered, both the left eye and the right eye are covered, and neither the left eye nor the right eye is covered.

According to a second aspect, the present application provides an apparatus for measuring a deviation angle of a gaze position based on 3D reconstruction.

The present application is implemented by the following technical solution:

The apparatus for measuring a deviation angle of a gaze position based on 3D reconstruction includes:
an image acquisition module configured to acquire video streaming information of a face of a testee, and obtain a face image sequence of the testee based on the video streaming information;
a key frame extraction module configured to input the face image sequence into a first neural network to obtain covering conditions of the face image sequence, and determine key frame images of the face image sequence based on the covering conditions;
a face 3D reconstruction module configured to input the key frame images into a second neural network to obtain feature point heat maps of the key frame images, and convert the feature point heat maps into facial feature point coordinates; and construct a 3D face model, obtain an objective function between the facial feature point coordinates and projection coordinates of the 3D face model based on the facial feature point coordinates and the 3D face model, and obtain a head pose corresponding to the facial feature point coordinates of the key frame images based on the objective function;
an eye 3D reconstruction module configured to select a reference gaze position image of a left eye and a reference gaze position image of a right eye from the key frame images to initialize an eyeball position, set an eyeball rotation angle of a reference gaze position as a preset angle based on the head pose, and solve reference 3D coordinates of eyeballs in the reference gaze position images in a head coordinate system; and
a deviation angle measurement module configured to fix 3D coordinates of the eyeball in a to-be-measured image in the head coordinate system based on the reference 3D coordinates, solve an eyeball rotation angle in the to-be-measured image, and obtain a deviation angle of a gaze position in the to-be-measured image based on the eyeball rotation angle in the to-be-measured image and the eyeball rotation angle of the reference gaze position.

According to a third aspect, the present application provides a computing device.

The present application is implemented by the following technical solution:

The computer device includes a memory, a processor, and a computer program stored in the memory and executable on the processor. The processor, when executing the computer program, implements any of the steps of the foregoing method for measuring a deviation angle of a gaze position based on 3D reconstruction.

According to a fourth aspect, the present application provides a computer-readable storage medium.

The present application is implemented by the following technical solution:

The computer-readable storage medium stores a computer program. The computer program, when executed by a processor, implements any of the steps of the foregoing method for measuring a deviation angle of a gaze position based on 3D reconstruction.

In summary, in comparison with the prior art, beneficial effects brought by the technical solutions provided in the embodiments of the present application at least include: The face image sequence of the testee is acquired. The covering conditions of the face image sequence are obtained through the first neural network. The key frame images are determined. The feature point heat maps are obtained through the second neural network and converted into the facial feature point coordinates. An objective function between a facial feature point and a projection point of the 3D face model is constructed. The head pose corresponding to the key frame images is obtained. The eyeball position is initialized. The eyeball rotation angle of the reference gaze position is set as the preset angle based on the head pose. The 3D coordinates of the eyeball in the reference gaze position image in the head coordinate system are solved. The 3D coordinates of the eyeball in the to-be-measured image in the head coordinate system are fixed. The eyeball rotation angle in the to-be-measured image is solved. The deviation angle of the gaze position is obtained. There is only a need to manually operate the image acquisition module to acquire appropriate video streaming information and transmit the data. Subsequent work is calculated and processed by each module stored in a cloud platform without limitation of hospital space. A user can quickly obtain an analysis result after uploading data through an intelligent device such as a mobile phone or a computer at any time and any place. This greatly shortens examination time and improves convenience. Acquired images are analyzed and processed through a neural network model. Subjective factors of a collector and a measured person have small impact such that measurement accuracy and consistency are improved.

### BRIEF DESCRIPTION OF THE DRAWINGS

The sole FIGURE is a schematic flowchart of a method for measuring a deviation angle of a gaze position based on 3D reconstruction according to an exemplary embodiment of the present application.

### DETAILED DESCRIPTION

This specific embodiment is merely an explanation of the present application, but it does not limit the present application. Those skilled in the art can make modifications without creative contribution to this embodiment as needed after reading this specification, but these modifications are protected by patent law as long as they fall within the scope of the claims of the present application.

To make the objectives, technical solutions, and advantages of the embodiments of the present application clearer, the following clearly and completely describes the technical solutions in the embodiments of the present application with reference to the accompanying drawings in the embodiments of the present application. Apparently, the described embodiments are some rather than all of the embodiments of the present application. All other embodiments obtained by those of ordinary skill in the art based on the embodiments of the present application without creative efforts shall fall within the protection scope of the present application.

In addition, the term “and/or” used in the present application merely describes an association relationship between associated objects, and indicates that three types of relationships may exist. For example, A and/or B may indicate that A exists alone, both A and B exist, or B exists alone. In addition, unless otherwise specified, the character “/” used in the present application generally indicates that the associated objects are in an “or” relationship.

Terms such as “first” and “second” in the present application are used to distinguish same or similar items that have a basically same effect and function. It should be understood that “first”, “second”, and “n^{th}” do not have a logical or temporal dependency relationship, nor do they limit a quantity and an execution order.

The embodiments of the present application are further described below in detail with reference to the accompanying drawings of this specification.

In an embodiment of the present application, a method for measuring a deviation angle of a gaze position based on 3D reconstruction is provided. As shown in the sole FIGURE, main steps are described as follows:

S10: Acquire video streaming information of a face of a testee, and obtain a face image sequence of the testee based on the video streaming information.

In some embodiments, a first visual target is disposed at a first preset distance and a second visual target is disposed at a second preset distance in front of the testee. A camera is used to acquire video streaming information of the testee gazing at the first visual target and the second visual target when the left eye is covered, the right eye is covered, both the left eye and the right eye are covered, and neither the left eye nor the right eye is covered.

Specifically, the head of the testee is first fixed through a head positioning device such that the lower jaw of the testee is placed on a jaw support, the forehead is tightly attached to a forehead band, and a position is appropriately adjusted to make a binocular line of sight of the testee be horizontal as far as possible. The first visual target is disposed at the first preset distance in front of the line of sight of the testee. The first preset distance is a distance between the first visual target and the head of the testee, and is 3-6 m. The first visual target is a far visual target. The second visual target is disposed at the second preset distance in front of the line of sight of the testee. The second preset distance is a distance between the second visual target and the head of the testee, and is 0.2-0.5 m. The second visual target is a near visual target. The first visual target and the second visual target are in a straight line. A high-definition camera is disposed coaxially with the first visual target and the second visual target. The high-definition camera is used to acquire the video streaming information of the testee gazing at the first visual target when only the left eye is covered by one baffle, only the right eye is covered by one baffle, both the left eye and the right eye are covered by two baffles, and neither the left eye nor the right eye is covered; and simultaneously acquire the video streaming information of the testee gazing at the second visual target when only the left eye is covered by one baffle, only the right eye is covered by one baffle, both the left eye and the right eye are covered by two baffles, and neither the left eye nor the right eye is covered. The face image sequence of the testee is obtained from the acquired video streaming information. The obtained face image sequence of the testee is a time period from the beginning of the test to the end of the test. For example, if a total length of the time period is 40 seconds and a video frame rate is 30 frames/second, a total of 40×30 face images are obtained as the face image sequence.

S20: Input the face image sequence into a first neural network to obtain covering conditions of the face image sequence, and determine key frame images of the face image sequence based on the covering conditions.

The first neural network is a deep residual network (ResNet). The obtained face image sequence of the testee is input into the ResNet such that the covering condition of each image can be obtained. The covering conditions are classified into 4 types: the left eye is covered, the right eye is covered, both the left eye and the right eye are covered, and neither the left eye nor the right eye is covered. The covering conditions of the face image sequence are sorted in chronological order, and images with a change in the covering condition are used as the key frame images. The key frame images are further classified into a reference gaze position, a manifest deviation gaze position, a total deviation gaze position, and the like based on different change conditions. The reference gaze position may also be referred to as a primary gaze position. A monocular gaze position is defined as a gaze position of a gaze eye in 3D coordinates after one eye is covered. The visual target may be directly in front of the face or in another direction that causes eye movement. The reference gaze position, the manifest deviation gaze position, and the total deviation gaze position are described by using a frontal gaze as an example.

Manifest deviation gaze position: an angle at which a squint eye deviates during a binocular gaze (one eye is a gaze eye and the other eye is the squint eye).

Total deviation gaze position: An alternate monocular gaze is formed by alternately covering one eye with an opaque baffle to break binocular alignment. In this case, the total deviation gaze position can be induced. For example, during alternate covering, the baffle covering the right eye is to be moved to cover the left eye, and a gaze position of the right eye when the baffle is just removed from the right eye is a total deviation gaze position of the right eye. (This is common knowledge in the industry.)

This technology calculates the reference gaze position, the manifest deviation gaze position, and the total deviation gaze position of each eye in pairs.

The reference gaze position, the manifest deviation gaze position, and the total deviation gaze position are calculated after the gaze at the first visual target (near vision). Then, the foregoing calculation is repeated after the gaze at the second visual target (far vision). Far vision first or near vision first may be determined based on actual clinical needs.

Changes in the covering condition may be classified into 12 types: a change from the condition in which neither the left eye nor the right eye is covered to the condition in which the left eye is covered, change from the condition in which neither the left eye nor the right eye is covered to the condition in which the right eye is covered, change from the condition in which neither the left eye nor the right eye is covered to the condition in which both the left eye and the right eye are covered, change from the condition in which the left eye is covered to the condition in which neither the left eye nor the right eye is covered, change from the condition in which the left eye is covered to the condition in which the right eye is covered, change from the condition in which the left eye is covered to the condition in which both the left eye and the right eye are covered, change from the condition in which the right eye is covered to the condition in which neither the left eye nor the right eye is covered, change from the condition in which the right eye is covered to the condition in which the left eye is covered, change from the condition in which the right eye is covered to the condition in which both the left eye and the right eye are covered, change from the condition in which both the left eye and the right eye are covered to the condition in which the left eye is covered, change from the condition in which both the left eye and the right eye are covered to the condition in which the right eye is covered, and change from the condition in which both the left eye and the right eye are covered to the condition in which neither the left eye nor the right eye is covered.

Preferably, the first neural network needs to be pre-trained to improve output accuracy of the first neural network. The pre-training is specifically as follows: Construct an initial first neural network, acquire a face image sequence, and label real covering conditions of the face image sequence; and sequentially input each image in the face image sequence as a training image into the initial first neural network to obtain a predicted covering condition of the training image, construct a cross-entropy loss function based on the predicted covering condition and a corresponding real covering condition, and perform supervised training on the initial first neural network based on the cross-entropy loss function to obtain a trained neural network as the first neural network. After each image is processed by the neural network, a predicted label is output. The predicted covering condition includes the predicted label. The predicted label includes: the left eye is covered, the right eye is covered, both the left eye and the right eye are covered, and neither the left eye nor the right eye is covered. A quantity of predicted labels is the same as a quantity of images in the face image sequence.

Specifically, the initial first neural network is constructed, and the obtained face image sequence is manually labeled with the real covering condition to obtain a face image sequence with a classification label of the real covering condition. Each image in the face image sequence is sequentially input into the untrained initial first neural network. The predicted covering condition of each image is obtained through recognition. The cross-entropy loss function is constructed based on the predicted covering condition and the real covering condition. The initial first neural network is trained based on the cross-entropy loss function to obtain the trained neural network as the first neural network for key frame extraction. The output accuracy of the first neural network can be improved through training with a large amount of data in an early stage. It is ensured that an output result of the first neural network can reach prediction accuracy (classification accuracy of the covering condition reaches more than 90% or even more than 95%). Accuracy of subsequently measuring a deviation angle of a gaze position can be further improved.

S30: Input the key frame images into a second neural network to obtain feature point heat maps of the key frame images, and convert the feature point heat maps into facial feature point coordinates.

A network model of the second neural network is a U-Net model. The face image sequence is input into the U-Net model such that N predicted feature point heat maps of the face image sequence can be obtained, where N is greater than 1.

Preferably, the network model of the second neural network is pre-trained to improve output accuracy of the network model of the second neural network. The pre-training is specifically as follows: Construct an initial second neural network. Obtain a face image from a public dataset. Label real feature point coordinates on the face image. Convert the real feature point coordinates into a real feature point heat map. Specifically, obtain a face image from a public face image database, process the face image, label N real feature point coordinates, label two-dimensional (2D) positions of a nose, a mouth, and irises, and perform sampling through Gaussian distribution to convert the N real feature point coordinates into N feature point heat maps. Input the face image into the initial second neural network to obtain a predicted feature point heat map of the face image. Construct an L1 loss function based on the real feature point heat map and the predicted feature point heat map. Perform supervised training on the initial second neural network based on the L1 loss function until the L1 loss function converges to obtain a trained initial second neural network as the second neural network.

After the second neural network is trained to preset accuracy (a normalized pixel error is at most 5%), the acquired face image sequence is input into the trained second neural network such that the feature point heat maps of the face image sequence can be obtained. Then, the feature point heat map is converted into the feature point coordinates, which are used to construct a 3D face model during subsequent 3D face reconstruction.

S40: Construct a 3D face model, construct an objective function between the facial feature point coordinates and projection coordinates of the 3D face model based on the facial feature point coordinates and the 3D face model, and obtain a head pose corresponding to the facial feature point coordinates of the key frame images based on the objective function.

Specifically, a parameterized 3D face model is first constructed by using 3D scan data of a plurality of persons and a plurality of expressions. An objective function between a facial feature point and a projection point of the 3D face model is constructed based on the facial feature point coordinates obtained in S30. A Euclidean distance between the points in 2D space is constrained to be short. The head pose is solved through an iterative optimization method. The iterative optimization method is a Gauss-Newton method or a conjugate gradient method.

S50: Select a reference gaze position image of a left eye and a reference gaze position image of a right eye from the key frame images to initialize an eyeball position. Set an eyeball rotation angle of a reference gaze position as a preset angle based on the head pose. Solve reference 3D coordinates of eyeballs in the reference gaze position images in a head coordinate system. The reference 3D coordinates are 3D coordinates of the eyeballs in the reference gaze position images in the head coordinate system. The head coordinate system is a coordinate system of the 3D face model.

Specifically, the reference gaze position images of the left eye and the right eye are selected based on the extracted key frame images and the facial feature point coordinates, to initialize the eyeball position. The initialization specifically includes the following steps: Set rotation angles of the eyeballs in the reference gaze position images as 0 degrees. Construct an objective function between an iris feature point and an iris model projection point. Solve the reference 3D coordinates of the eyeballs in the head coordinate system through iterative optimization. Iris model projection is projection of an iris model in a coordinate system in which the facial feature point is located.

S60: Fix 3D coordinates of the eyeball in a to-be-measured image in the head coordinate system based on the reference 3D coordinates, solve an eyeball rotation angle in the to-be-measured image, and obtain a deviation angle of a gaze position in the to-be-measured image based on the eyeball rotation angle in the to-be-measured image and the eyeball rotation angle of the reference gaze position.

Deviation angles of a manifest deviation gaze position and a total deviation gaze position can be solved based on the extracted key frame images and the facial feature point coordinates.

Herein, images of the manifest deviation gaze position and the total deviation gaze position in the key frame images are used as to-be-measured images. It should be noted that the to-be-measured images may be all images in the obtained face image sequence. The solving specifically includes the following steps: Fix the 3D coordinates of the eyeball in the to-be-measured image in the head coordinate system based on the obtained reference 3D coordinates of the reference gaze position. Construct the objective function between the iris feature point and the iris model projection point. Solve the eyeball rotation angle in the to-be-measured image through iterative optimization. Obtain the deviation angle of the gaze position in the to-be-measured image by subtracting the eyeball rotation angle in the to-be-measured image from the eyeball rotation angle of the reference gaze position.

In some embodiments, the method for measuring a deviation angle of a gaze position based on 3D reconstruction further includes: Construct a regression model of eyeball 3D rotation and a triangular prism deviation angle based on triangular prism deviation angles corresponding to video streaming data of a large quantity of face images of standardized squint and normal people acquired in the early stage. A corresponding triangular prism deviation angle can be obtained after a new face image sample is input into the regression model. The regression model can further assist in classification of esotropia, exotropia, vertical squint, recessive squint, and normal. With reference to clinical information, the regression model can assist in providing diagnosis and treatment decision-making opinions, including but not limited to risk assessment of amblyopia and suggestions for wearing glasses or surgery.

The present application further provides an apparatus for measuring a deviation angle of a gaze position based on 3D reconstruction. The apparatus includes:
an image acquisition module configured to acquire video streaming information of a face of a testee, and obtain a face image sequence of the testee based on the video streaming information, where the image acquisition module may be, for example, a high-definition camera, a 3D camera, a camera, or a webcam;
a key frame extraction module configured to input the face image sequence into a first neural network to obtain covering conditions of the face image sequence, and determine key frame images of the face image sequence based on the covering conditions, where the key frame extraction module may be, for example, a graphics processing unit (GPU), a central processing unit (CPU), or a device equipped with a CPU and a GPU, such as a server, a personal computer, or a smartphone;
a face 3D reconstruction module configured to input the key frame images into a second neural network to obtain feature point heat maps of the key frame images, and convert the feature point heat maps into facial feature point coordinates; and construct a 3D face model, construct an objective function between the facial feature point coordinates and projection coordinates of the 3D face model based on the facial feature point coordinates and the 3D face model, and obtain a head pose corresponding to the facial feature point coordinates of the key frame images based on the objective function, where the face 3D reconstruction module may be, for example, a CPU, or a device equipped with a CPU, such as a server, a personal computer, or a smartphone;
an eye 3D reconstruction module configured to select a reference gaze position image of a left eye and a reference gaze position image of a right eye from the key frame images to initialize an eyeball position, set an eyeball rotation angle of a reference gaze position as a preset angle based on the head pose, and solve reference 3D coordinates of eyeballs in the reference gaze position images in a head coordinate system, where the eye 3D reconstruction module may be, for example, a CPU, or a device equipped with a CPU, such as a server, a personal computer, or a smartphone; and
a deviation angle measurement module configured to fix 3D coordinates of the eyeball in a to-be-measured image in the head coordinate system based on the reference 3D coordinates, solve an eyeball rotation angle in the to-be-measured image, and obtain a deviation angle of a gaze position in the to-be-measured image based on the eyeball rotation angle in the to-be-measured image and the eyeball rotation angle of the reference gaze position, where the deviation angle measurement module may be, for example, a CPU, or a device equipped with a CPU, such as a server, a personal computer, or a smartphone.

There is only a need to manually operate the image acquisition module to acquire appropriate video streaming information and transmit the data. Subsequent work is calculated and processed by each module stored in a cloud platform without limitation of hospital space. A user can quickly obtain an analysis result after uploading data through an intelligent device such as a mobile phone or a computer at any time and any place. This greatly shortens examination time. In addition, subjective factors of a collector and a measured person have small impact such that measurement accuracy and consistency are high.

In an embodiment, a computer device is provided. The computer device may be a server.

The computer device includes a processor, a memory, a network interface, and a database that are connected through a system bus. The processor of the computer device is configured to provide calculation and control capabilities. The memory of the computer device includes a nonvolatile storage medium and an internal memory. The nonvolatile storage medium stores an operating system, a computer program, and the database. The internal memory provides an environment for operation of the operating system and the computer program in the nonvolatile storage medium. The network interface of the computer device is configured to communicate with an external terminal through a network. The computer program, when executed by the processor, implements any of the foregoing method for measuring a deviation angle of a gaze position based on 3D reconstruction.

In an embodiment, a computer-readable storage medium is provided, including a memory, a processor, and a computer program stored in the memory and executable on the processor. The processor executes the computer program to implement any of the foregoing method for measuring a deviation angle of a gaze position based on 3D reconstruction.

Those of ordinary skill in the art may understand that all or some of the procedures in the method of the foregoing embodiments may be implemented by a computer program instructing related hardware. The computer program may be stored in a nonvolatile computer-readable storage medium. When the computer program is executed, the procedures in the embodiments of the foregoing method may be performed. Any reference to a memory, a storage, a database, or other mediums used in various embodiments provided in the present application may include a nonvolatile memory and/or a volatile memory. The nonvolatile memory may include a read-only memory (ROM), a programmable ROM (PROM), an electrically programmable ROM (EPROM), an electrically erasable programmable ROM (EEPROM), or a flash memory. The volatile memory may include a random access memory (RAM) or an external cache memory. As description rather than limitation, the RAM may be obtained in a plurality of forms, such as a static RAM (SRAM), a dynamic RAM (DRAM), a synchronous DRAM (SDRAM), a double data rate SDRAM (DDRSDRAM), an enhanced SDRAM (ESDRAM), a synchronization link (synchlink) DRAM (SLDRAM), a Rambus direct RAM (RDRAM), a direct Rambus dynamic RAM (DRDRAM), and a Rambus dynamic RAM (RDRAM).

It may be clearly understood by a person skilled in the art that, for convenient and concise description, division into the foregoing functional units or modules is merely used as an example for illustration. In actual application, the foregoing functions may be allocated to different functional units or modules and implemented according to a requirement. That is, an inner structure of the system in the present application is divided into different functional units or modules to implement all or some of the foregoing functions.

1. A method for measuring a deviation angle of a gaze position based on three-dimensional (3D) reconstruction, comprising:
acquiring video streaming information of a face of a testee, and obtaining a face image sequence of the testee based on the video streaming information;
inputting the face image sequence into a first neural network to obtain covering conditions of the face image sequence, and determining key frame images of the face image sequence based on the covering conditions;
inputting the key frame images into a second neural network to obtain feature point heat maps of the key frame images, and converting the feature point heat maps into facial feature point coordinates;
constructing a 3D face model, obtaining an objective function between the facial feature point coordinates and projection coordinates of the 3D face model based on the facial feature point coordinates and the 3D face model, and obtaining a head pose corresponding to the facial feature point coordinates of the key frame images based on the objective function;
selecting a reference gaze position image of a left eye and a reference gaze position image of a right eye from the key frame images to initialize an eyeball position, setting an eyeball rotation angle of a reference gaze position as a preset angle based on the head pose, and solving reference 3D coordinates of an eyeball in the reference gaze position images in a head coordinate system; and
fixing 3D coordinates of the eyeball in a to-be-measured image in the head coordinate system based on the reference 3D coordinates and solving an eyeball rotation angle in the to-be-measured image; and obtaining a deviation angle of a gaze position in the to-be-measured image based on the eyeball rotation angle in the to-be-measured image and the eyeball rotation angle of the reference gaze position.

2. The method for measuring a deviation angle of a gaze position based on 3D reconstruction according to claim 1, before the inputting the face image sequence into a first neural network to obtain covering conditions of the face image sequence, further comprising:
training the first neural network, which specifically comprises: constructing an initial first neural network, acquiring a face image sequence, and labeling real covering conditions of the face image sequence; and sequentially inputting each image in the face image sequence as a training image into the initial first neural network to obtain a predicted covering condition of the training image, constructing a cross-entropy loss function based on the predicted covering condition and a corresponding real covering condition, and performing supervised training on the initial first neural network based on the cross-entropy loss function to obtain a trained neural network as the first neural network.

3. The method for measuring a deviation angle of a gaze position based on 3D reconstruction according to claim 1, before the inputting the key frame images into a second neural network to obtain feature point heat maps of the key frame images, further comprising:
training the second neural network, which specifically comprises: constructing an initial second neural network, obtaining a face image from a public dataset, labeling real feature point coordinates on the face image, and converting the real feature point coordinates into a real feature point heat map; and inputting the face image into the initial second neural network to obtain a predicted feature point heat map of the face image, constructing an L1 loss function based on the real feature point heat map and the predicted feature point heat map, and performing supervised training on the initial second neural network based on the L1 loss function until the L1 loss function converges to obtain a trained initial second neural network as the second neural network.

4. The method for measuring a deviation angle of a gaze position based on 3D reconstruction according to claim 1, wherein the fixing 3D coordinates of the eyeball in a to-be-measured image in the head coordinate system based on the reference 3D coordinates and solving an eyeball rotation angle in the to-be-measured image comprises:
fixing the 3D coordinates of the eyeball in the to-be-measured image in the head coordinate system, constructing an objective function between an iris feature point and an iris model projection point, and solving the eyeball rotation angle in the to-be-measured image through iterative optimization.

5. The method for measuring a deviation angle of a gaze position based on 3D reconstruction according to claim 1, wherein the eyeball rotation angle of the reference gaze position is set as the preset angle, and the preset angle is set to 0 degrees.

6. The method for measuring a deviation angle of a gaze position based on 3D reconstruction according to claim 1, wherein the acquiring video streaming information of a face of a testee comprises:
disposing a first visual target at a first preset distance and a second visual target at a second preset distance in front of the testee, and acquiring, through a camera, video streaming information of the testee gazing at the first visual target and the second visual target when the left eye is covered, the right eye is covered, both the left eye and the right eye are covered, and neither the left eye nor the right eye is covered.

7. The method for measuring a deviation angle of a gaze position based on 3D reconstruction according to claim 6, wherein the covering condition of the face image sequence is one of the following conditions: the left eye is covered, the right eye is covered, both the left eye and the right eye are covered, and neither the left eye nor the right eye is covered.

8. An apparatus for measuring a deviation angle of a gaze position based on three-dimensional (3D) reconstruction, comprising:
an image acquisition module configured to acquire video streaming information of a face of a testee, and obtain a face image sequence of the testee based on the video streaming information;
a key frame extraction module configured to input the face image sequence into a first neural network to obtain covering conditions of the face image sequence, and determine key frame images of the face image sequence based on the covering conditions;
a face 3D reconstruction module configured to input the key frame images into a second neural network to obtain feature point heat maps of the key frame images, and convert the feature point heat maps into facial feature point coordinates; and construct a 3D face model, obtain an objective function between the facial feature point coordinates and projection coordinates of the 3D face model based on the facial feature point coordinates and the 3D face model, and obtain a head pose corresponding to the facial feature point coordinates of the key frame images based on the objective function;
an eye 3D reconstruction module configured to select a reference gaze position image of a left eye and a reference gaze position image of a right eye from the key frame images to initialize an eyeball position, set an eyeball rotation angle of a reference gaze position as a preset angle based on the head pose, and solve reference 3D coordinates of an eyeball in the reference gaze position images in a head coordinate system; and
a deviation angle measurement module configured to fix 3D coordinates of the eyeball in a to-be-measured image in the head coordinate system based on the reference 3D coordinates, solve an eyeball rotation angle in the to-be-measured image, and obtain a deviation angle of a gaze position in the to-be-measured image based on the eyeball rotation angle in the to-be-measured image and the eyeball rotation angle of the reference gaze position.

9. A computer device, comprising a memory, a processor, and a computer program stored in the memory, wherein the processor executes the computer program to implement the steps of the method according to claim 1.

10. A computer device, comprising a memory, a processor, and a computer program stored in the memory, wherein the processor executes the computer program to implement the steps of the method according to claim 2.

11. A computer device, comprising a memory, a processor, and a computer program stored in the memory, wherein the processor executes the computer program to implement the steps of the method according to claim 3.

12. A computer device, comprising a memory, a processor, and a computer program stored in the memory, wherein the processor executes the computer program to implement the steps of the method according to claim 4.

13. A computer device, comprising a memory, a processor, and a computer program stored in the memory, wherein the processor executes the computer program to implement the steps of the method according to claim 5.

14. A computer device, comprising a memory, a processor, and a computer program stored in the memory, wherein the processor executes the computer program to implement the steps of the method according to claim 6.

15. A computer device, comprising a memory, a processor, and a computer program stored in the memory, wherein the processor executes the computer program to implement the steps of the method according to claim 7.

16. A computer-readable storage medium, wherein the computer-readable storage medium stores a computer program, and the computer program, when executed by a processor, implements the steps of the method according to claim 1.

17. A computer-readable storage medium, wherein the computer-readable storage medium stores a computer program, and the computer program, when executed by a processor, implements the steps of the method according to claim 2.

18. A computer-readable storage medium, wherein the computer-readable storage medium stores a computer program, and the computer program, when executed by a processor, implements the steps of the method according to claim 3.

19. A computer-readable storage medium, wherein the computer-readable storage medium stores a computer program, and the computer program, when executed by a processor, implements the steps of the method according to claim 4.

20. A computer-readable storage medium, wherein the computer-readable storage medium stores a computer program, and the computer program, when executed by a processor, implements the steps of the method according to claim 5.
